(19) United States
(12) Patent Application Publication
(10) Pub. No.: US 2008/0254005 A1
(43) Pub. Date: Oct. 16, 2008

(54) Stem Cell Therapy for the Treatment of Autism and Other Disorders

(75) Inventors: Neil H. Riordan, Chandler, AZ (US); Thomas E. Ichim, San Diego, CA (US)

Correspondence Address: BAUMGARTNER PATENT LAW, 5933 N.E. WIN SIVERS DR. SUITE 250, PORTLAND, OR, 97220, US

(73) Assignee: Medistem Labortories, Scottsdale, AZ (US)

(21) Appl. No.: 12/098,420

(22) Filed: Apr. 5, 2008

Related U.S. Application Data
(60) Provisional application No. 60/910,605, filed on Apr. 6, 2007.

(51) Int. Cl. A61K 35/12; A61P 43/00

(52) U.S. Cl. 424/93.7

(57) ABSTRACT

Disclosed are methods, compositions of matter, and cells, useful for the treatment of autism, social integrative disorders, and various cognitive abnormalities. The invention discloses, inter alia, means of inducing angiogenesis and immune modulation either in sequence or parallel in order to substantially ameliorate or reverse the progression of autism. The use of stem cells, and cells naturally possessing or endowed with angiogenic and anti-inflammatory activity are disclosed for autism either alone or in combination with various therapeutic interventions.

### CROSS-REFERENCE TO RELATED APPLICATIONS

This application claims priority to Provisional Application Ser. No. 60/910,605, filed Apr. 6, 2007, and entitled “Stem Cell Therapy for Autism” which is hereby expressly incorporated by reference in its entirety.

### FIELD OF THE INVENTION

The invention relates to the area of neurological and behavioral disorders. More particularly it relates to intervention in the disorder of autism through modification of two major features of autism: inflammation and neural hypoperfusion.

### BACKGROUND

Abnormal immune activity has been implicated in the neurodevelopment disorder of autism spectrum disorder (ASD). A recent study of a cohort of children with ASD demonstrated and ASD-associated systemic and intestinal immune dysregulation similar to that found in Crohn's disease (CD). CD is a chronic inflammatory disorder of the gastrointestinal tract driven by activated type 1 helper T-cells which results from a deregulated mucosal immune response to normal constituents of the gut microflora. (World J Gastroenterol. 2006 Sep. 21; 12(35):5606-10. Interleukin-12 and Th1 immune response in Crohn's disease: pathogenetic relevance and therapeutic implication. Peluso I, Pallone F, Monteleone G.)

In particular peripheral blood and mucosal CD3+ TNFalpha+ and CD3+ IFNgamma+ cells were significantly increased in children with ASD versus non-inflamed controls. (J Neuroimmunol. 2006 April; 173(1-2):126-34. Epub 2006 Feb. 21. Immune activation of peripheral blood and mucosal CD3+ lymphocyte cytokine profiles in children with autism and gastrointestinal symptoms. Ashwood P, Wakefield A J.) Significantly higher plasma Th1 cytokines IL-12 and IFN-gamma have been found patients with ASD compared to age-matched controls. (J Neuroimmunol. 1996 May; 66(1-2):143-5 Plasma increase of interleukin-12 and interferon-gamma. Pathological significance in autism. Singh V K.)

In addition to immune dysregulation, ASD patients have been shown to have decreased blood flow to the brain. Two studies have identified and describe the phenomenon of temporal hypoperfusion in ASD children. The temporal regions of the brain are responsible for the types of impairments send in autism: language, social perception, and “theory of mind.” A recent study demonstrated a significant negative correlation between cerebral blood flow (RCBF) measured at rest, and Autism Diagnostic Interview-Revised (ADI-R) scores. The conclusion being, the more severe the autistic syndrome, the more RCBF is low in this region, suggesting that left superior temporal hypoperfusion is related to autistic behavior severity. (Ann Neurol. 2005 September; 58(3):466-9. Autism severity and temporal lobe functional abnormalities Gendry Meresse I.)

Children with ASD have had positive clinical responses to treatment with non-matched infusions of CD34+ stem cells from umbilical cord. These cells are pro-angiogenic and are known to preferentially accumulate at areas of relative hypoxia. They have been used successfully in restoring blood flow in the lower extremities of patients with critical limb ischemia.

Mesenchymal stem cells are known to suppress T-cell immunity. There are two current clinical trials of expanded, unmatched, bone marrow-derived mesenchymal stem cells in the treatment of Graft versus host disease, and Crohn's disease. The Crohn's disease trial is in Phase III and has been given fast-tract status by the US FDA. (www.celltherapynews.com/index.cfm?act=nl&do=newsletter&nl_ID=198&yr=2007&m nth=1 Osiris Therapeutics Receives FDA Fast Track Status for Its Crohn's Disease Stem Cell Therapy and Clearance to Start Phase III Clinical Trial: Osiris Therapeutics, Inc. announced today that PROCHYMAL™ has received Fast Track designation from the U.S. Food and Drug Administration, expediting the development of the stem cell treatment for Crohn's Disease that does not respond to standard therapies.) There have been positive results in the treatment of both diseases.

The inherent safety of non-related, unmatched CD34+ cells is known. Expanded allogeneic mesenchymal cells are widely known to be immune privileged and are being used in at least 4 clinical trials in the US at this time.

### SUMMARY OF THE INVENTION

In one embodiment, the invention provides a method of treating a pervasive developmental disorder that includes providing: a) a cell capable of inhibiting inflammatory responses, b) a cell capable of stimulating angiogenesis; and c) administration of said cell described in a) and said cell described in b) either in sequence or concurrently into a patient in need thereof.

In one embodiment mesenchymal stem cells are used as cells capable of inhibiting inflammatory responses. Said mesenchymal stem cells may be derived from sources selected from a group comprising of: peripheral blood, mobilized peripheral blood, bone marrow, menstrual blood, endometrial aspiration, adipose tissue, deciduous teeth, Wharton's jelly, placental matrix, cord blood, and peripheral tissue. Additionally mesenchymal stem cells may be derived from embryonic stem cells.

In one embodiment, cells of the immune system are used for the suppression of inflammatory reactions. Said cells of the immune system may be selected from a group comprising of: a T regulatory cell, a T suppressor cell, an alternatively activated macrophage, a tolerogenic dendritic cells, a lymphoid dendritic cells, and an immature dendritic cell.

In one embodiment, cells capable of inducing an anti-inflammatory effect are activated in vivo through administration of an agent or plurality of agents known to activate anti-inflammatory activities of said cells. Said agents capable of activating anti-inflammatory activities are selected from a group comprising of: activators of immune suppressive properties, and inhibitors of inflammatory agents, wherein said activators of immune suppressive properties are selected from a group comprising of: IL-4, IL-10, IL-13, IL-20, TGF-alpha, TGF-beta, VEGF, and IFN-omega. Furthermore inhibitors of inflammatory agents may be selected from a group comprising of: antibodies to inflammatory mediators, blocking proteins to inflammatory mediators, soluble receptors of inflammatory mediators, small molecule receptor antagonists to inflammatory mediators, nucleic acid aptamers to inflammatory mediators, antisense oligonucleotides to inflammatory mediators, and inducers of RNA interference to inflammatory mediators. Said inhibitors of inflammatory agents may be a decoy oligonucleotide that substantially inhibits binding of a transcription factor associated with inflammation to its natural DNA target sequence.

In one embodiment said anti-inflammatory activity may be endowed on said cells through culture in conditions known to upregulate expression of mediators known to inhibit inflammation. Said conditions may be selected from a group comprising of culture in: IL-10, IL-4, IL-13, IL-20, TGF-alpha, TGF-beta, VEGF, and IFN-omega.

In one embodiment said anti-inflammatory activity is endowed on said cells through transfection with genes whose protein products are known to inhibit inflammation. Said transfected genes may be selected from a group comprising of culture in: IL-10, IL-4, IL-13, IL-20, TGF-alpha, TGF-beta, VEGF, and IFN-omega.

In one embodiment cells capable of stimulating angiogenesis are utilized for the practice of the invention, said cells may acts via differentiation into endothelial cells. Alternatively, said cells capable of stimulating angiogenesis may act via differentiation into non-endothelial cells associated with the vasculature. Alternatively said cell capable of stimulating angiogenesis acts via production of mediators that stimulate angiogenesis.

In one embodiment said cell capable of stimulating angiogenesis is a stem cell, an endothelial progenitor cell, a bone marrow mononuclear cell, a cord blood derived mononuclear cell, a CD34 cell, or a CD133 cell.

In one embodiment an angiogenic agent is utilized instead of a cell in order to induce angiogenesis. Said angiogenic agent may be selected from a group comprising of: VEGF and various isoforms and family members, FGF and various isoforms and family members, TGF and various isoforms and family members, HGF and various isoforms and family members, and adrenomedulin. Additionally, said angiogenic agent may be a culture supernatant or an extract of angiogenic activity purified from a culture supernatant.

In one embodiment culture supernatant includes culture of live tissue, for example, said culture supernatant may include perfusate of a live placenta, or otherwise known as live placentally conditioned media.

In one embodiment said culture supernatant or an extract of angiogenic activity is purified from a culture supernatant and is derived from a culture of cells under hypoxic conditions.

In one embodiment said culture supernatant or an extract of angiogenic activity purified from a culture supernatant is derived from a culture of bone marrow cells, cord blood cells, or stem cells. Said stem cells may be selected from a group comprising of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

Further embodiments relate to methods of treating a pervasive developmental disorder comprising: providing a) a first cell capable of inhibiting inflammation and b) a second cell capable of stimulating angiogenesis; and administering said first and second cells either in sequence or concurrently into a patient in need thereof. Said pervasive developmental disorder can be selected from the group consisting of: Autism, Rett's Disorder, Childhood Disintegrative Disorder, and Asperger's Syndrome, and others, for example.

A preferred cell capable of inhibiting inflammation is a mesenchymal stem cell, for example. A preferred cell capable of inhibiting inflammation is activated in vivo by administering one or more agents capable of activating an anti-inflammatory response in said cell to the patient in need. An advantageous cell capable of stimulating angiogenesis can act by differentiating into cells of the patient's vasculature or by providing trophic support to cells of the patient's vasculature.

Preferred methods herein include cells capable of stimulating angiogenesis that are selected from the group consisting of: a) a cord blood derived CD34 cell, b) a cord blood mononuclear cell, c) a placental matrix mesenchymal cell, d) a mesenchymal cell, e) an endothelial progenitor cell, f) a monocytic cell, g) a bone marrow derived CD34 cell, h) a cord blood derived CD133 cell, and i) a bone marrow derived CD133 cell, and the like, for example.

Further methods can include administering an angiogenic agent to the patient in order to augment the angiogenic activity of said cell capable of stimulating angiogenesis.

Additional embodiments relate to methods of treating a pervasive developmental disorder that include concurrently stimulating an anti-inflammatory process and a pro-angiogenic process in a patient in need thereof. For example, stimulating an anti-inflammatory process can be accomplished by administering a mesenchymal stem cell to said patient and stimulating a pro-angiogenic process can be accomplished by administering a cord blood derived CD34 cell to said patient. Additionally, the stimulation of an anti-inflammatory process can be accomplished by the administration of a non-cellular pharmaceutical anti-inflammatory agent. Furthermore, the stimulation of a pro-angiogenic process can be accomplished by administering a pro-angiogenic, non-cellular pharmaceutical. Additionally, a combination of an anti-inflammatory non-cellular pharmaceutical can be provided with a pro-angiogenic cell.

Further embodiments relate to kits that are useful for the treatment of a pervasive developmental disorder in a patient in need thereof and include a) a cell capable of stimulating angiogenesis in said patient; and b) a cell capable of inhibiting inflammation in said patient. A preferred cell capable of stimulating angiogenesis is a cord blood derived CD34 cell. A preferred cell capable of inhibiting inflammation is a mesenchymal stem cell. Advantageously the mesenchymal stem cell is collected from a source selected from the group consisting of: a) bone marrow, b) placental matrix, c) adipose tissue, d) menstrual blood, e) endometrium, f) muscle, g) circulating blood, and h) cord blood, for example. More specifically, the cell capable of stimulating angiogenesis and/or the cell capable of inhibiting inflammation (e.g., mesenchymal stem cell) are allogeneic. The kit can further include instructions for administering said cell capable of stimulating angiogenesis and said cell capable of inhibiting inflammation to treat said pervasive developmental disorder in said patient.

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

The invention revolves around the concept that treatment of autism and autism spectrum disorders can be performed by ameliorating two main pathological features of this condition: hypoperfusion of specific areas of the brain, and inflammatory responses. The invention teaches that by either sequentially, or concurrently inhibiting these processes it is feasible to induce reversal of the disease.

In one embodiment a patient with autism is treated by concurrent administration of CD34+ umbilical cord stem cells and mesenchymal stem cells. The treatment is performed with the aim of the CD34+ cells homing to the hypoperfused area of the brain and subsequently causing stimulation of angiogenesis and ultimately therefore decreasing hypoperfusion. The mesenchymal cells are incorporated into the treatment in order to inhibit the Th1 immune dysregulation systemically, and/or in some cases, specifically in the gut.

In another embodiment, an exogenous angiogenic agent is administered systemically together with mesenchymal stem cells, in this embodiment the mesenchymal stem cells inhibit inflammatory processes, whereas the exogenously administered angiogenic agent stimulates angiogenesis in order to increase perfusion. The use of exogenous angiogenic agents is preferably, but not exclusively, limited to agents that have specific activity on hypoxic tissue. In this manner angiogenesis will be limited to the area of hypoperfusion. Agents that selectively induce angiogenesis in areas of hypoperfusion include factors such as members of the FGF family whose receptors are upregulated in areas of tissue hypoxia.

In another embodiment angiogenesis stimulatory cells are provided together with an exogenous immune modulator. Such exogenous immune modulators may have anti-inflammatory activity such as IL-10, IL-4, or TGF family members. Other anti-inflammatory agents useful for the practice of this invention will be obvious to one of skill in the art. Examples of clinically used anti-inflammatory agents include: Alclofenac; Alclometasone Dipropionate; Algestone Acetonide; Alpha Amylase; Alpha-lipoic acid; Alpha tocopherol; Amcinafal; Amcinafide; Amfenac Sodium; Amiprilose Hydrochloride; Anakinra; Anirolac; Anitrazafen; Apazone; Ascorbic Acid; Balsalazide Disodium; Bendazac; Benoxaprofen; Benzydamine Hydrochloride; Bromelains; Broperamole; Budesonide; Carprofen; Chlorogenic acid; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Ellagic acid; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Glutathione; Halcinonide; Halobetasol Propionate; Halopredone Acetate; Hesperedin; Tbufenac; Tbuprofen; Tbuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Lycopene; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Oleuropein; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Pycnogenol; Polyphenols; Prednazate; Prifelone; Prodolic Acid; Proquazone; Proxazole; Proxazole Citrate; Quercetin; Reseveratrol; Rimexolone; Romazarit; Rosmarinic acid; Rutin; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrahydrocurcumin; Tetrydamine; Tiopinac; Tixocortol Pivalate; Tolmetin; Tolmetin Sodium; Triclonide; Triflumidate; Zidometacin; and Zomepirac Sodium. Angiogenesis stimulatory cells that may be utilized in combination with anti-inflammatory agents include stem cells that intrinsically are angiogenic, or that have been endowed with angiogenic potential. Such stem cells may be derived from embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells. Angiogenic potential may be endowed by gene transfection with agents stimulatory of angiogenesis, culture in endothelial differentiation media, culture with agents known to induce angiogenesis, or culture under conditions of hypoxia.

In one embodiment, mesenchymal stem cells are provided in absence of cord blood stem cells with the purpose that mesenchymal stem cells will inhibit inflammation and as a result ameliorate autism.

Mesenchymal stem cells may be derived from sources selected from a group comprising of: peripheral blood, mobilized peripheral blood, bone marrow, menstrual blood, endometrial aspiration, adipose tissue, deciduous teeth, Wharton's jelly, placental matrix, cord blood, and peripheral tissue.

In one embodiment, cord blood CD34 cells are provided in absence of mesenchymal stem cells with the purpose that CD34 cord blood cells will induce angiogenesis and contribute to amelioration of autism.

In one embodiment allogeneic endometrial regenerative cells are collected from the menstrual blood and expanded in vitro as described in Meng et al. Endometrial regenerative cells: a novel stem cell population. J Transl Med. 2007 Nov. 15; 5:57. Said cells are generated under Good Manufacturing Practices and released according to release criteria demanding sterility, purity and functionality. Cells are administered intravenously into patients possessing autism associated inflammatory changes. Said cells are administered at a dose and frequency sufficient to inhibit local inflammatory changes found in the gastrointestinal tract of patients. In some cases patients are treated with doses ranging from 1-100 million cells intravenously. More preferred, patients are treated with 1-10 million cells intravenously. Yet more preferred patients are treated with approximately 5 million cells intravenously three times every second day. In another embodiment, mesenchymal stem cells are derived from the cord blood, placental matrix, amniotic fluid, bone marrow, or peripheral blood. Derivation of mesenchymal stem cells is well known in the art and has been described in several publications for example (Sun et al. In Vitro Proliferation and Differentiation of Human Mesenchymal Stem Cells Cultured in Autologous Plasma Derived from Bone Marrow. Tissue Eng Part A. 2008 March; 14(3):391-400; Ball et al. Cotransplantation of ex vivo expanded mesenchymal stem cells accelerates lymphocyte recovery and may reduce the risk of graft failure in haploidentical hematopoietic stem-cell transplantation. Blood. 2007 Oct. 1; 110(7):2764-7; Schuleri et al. Mesenchymal stem cells for cardiac regenerative therapy. Handb Exp Pharmacol. 2007; (180):195-218).

### EXAMPLES

### Example 1

### Autologous Cord Blood Therapy

20 children meeting the DSM-IV, ADI-R and ADOS-G criteria for autistic disorder are recruited into an experimental study. 10 patients serve as placebo controls whereas 10 receive active treatment, including the administration of autologous cord blood mononuclear cells. The Aberrant Behavior Checklist (ABC) score and the Vineland Adaptive Behavior Scale are used in the selection of patients to enable the study to compare groups with similar characteristics.

Autologous cord blood administration is performed according to conventional medical practice, specifically, umbilical cord blood is purified according to routine methods ((Rubinstein, et al. Processing and cryopreservation of placental/umbilical cord blood for unrelated bone marrow reconstitution. Proc Natl Acad Sci USA 92:10119-10122). Briefly, a 16-gauge needle from a standard Baxter 450-ml blood donor set containing CPD A anticoagulant (citrate/phosphate/dextrose/adenine) (Baxter Health Care, Deerfield, Ill.) is inserted and used to puncture the umbilical vein of a placenta obtained from healthy delivery from a mother tested for viral and bacterial infections according to international donor standards. Cord blood is allowed to drain by gravity so as to drip into the blood bag. The placenta is placed in a plastic-lined, absorbent cotton pad suspended from a specially constructed support frame in order to allow collection and reduce the contamination with maternal blood and other secretions. The 63 ml of CPD A used in a standard blood transfusion bag, calculated for 450 ml of blood, is reduced to 23 ml by draining 40 ml into a graduated cylinder just prior to collection. This volume of anticoagulant matches better than the cord volumes usually retrieved (<170 ml).

An aliquot of the blood is removed for safety testing according to the standards of the National Marrow Donor Program (NMDP) guidelines. Safety testing includes routine laboratory detection of human immunodeficiency virus 1 and 2, human T-cell lymphotropic virus I and II, Hepatitis B virus, Hepatitis C virus, Cytomegalovirus and Syphilis. Subsequently, 6% (wt/vol) hydroxyethyl starch is added to the anticoagulated cord blood to a final concentration of 1.2%. The leukocyte rich supernatant is then separated by centrifuging the cord blood hydroxyethyl starch mixture in the original collection blood bag (50×g for 5 min at 10° C.). The leukocyte-rich supernatant is expressed from the bag into a 150-ml Plasma Transfer bag (Baxter Health Care) and centrifuged (400×g for 10 min) to sediment the cells. Surplus supernatant plasma is transferred into a second plasma Transfer bag without severing the connecting tube. Finally, the sedimented leukocytes are resuspended in supernatant plasma to a total volume of 20 ml. Approximately 5×10⁸-7×10⁹ nucleated cells are obtained per cord. Cells are cryopreserved according to the method described by Rubinstein, et al. (Rubinstein, et al. Processing and cryopreservation of placental/umbilical cord blood for unrelated bone marrow reconstitution. Proc Natl Acad Sci USA 92:10119-10122) for subsequent cellular therapy. At the time of infusion, cells are thawed and assessed for viability and purity according to the method published by Rubinstein, et al. Cells are washed and concentrated to a volume of 10 ml in UPS saline with 5% autologous serum. Cells are administered intravenously.

A statistically significant response in The Aberrant Behavior Checklist (ABC) score and the Vineland Adaptive Behavior Scale is observed in the patients receiving autologous cord blood but not placebo at the 1 month follow-up point.

### Example 2

### Allogeneic Cord Blood with Allogeneic Mesenchymal Stem Cells 20 children meeting the DSM-IV, ADI-R and ADOS-G criteria for autistic disorder are recruited into an experimental study. 10 patients serve as placebo controls whereas 10 receive active treatment, including the administration of allogeneic expanded cord blood CD34 and allogeneic mesenchymal stem cells. The Aberrant Behavior Checklist (ABC) score and the Vineland Adaptive Behavior Scale are used in the selection of patients to enable the study to compare groups with similar characteristics.

Cord blood cells are purified according to methods of Example I and CD34 cells are expanded by culture. CD34+ cells are purified from the mononuclear cell fraction by immuno-magnetic separation using the Magnetic Activated Cell Sorting (MACS) CD34+ Progenitor Cell Isolation Kit (Miltenyi-Biotec, Auburn, Calif.) according to manufacturer's recommendations. The purity of the CD34+ cells obtained ranges between 95% and 98%, based on Flow Cytometry evaluation (FACScan flow cytometer, Becton-Dickinson, Immunofluorometry systems, Mountain View, Calif.). Cells are plated at a concentration of 10.sup.4 cells/ml in a final volume of 0.5 ml in 24 well culture plates (Falcon; Becton Dickinson Biosciences) in DMEM supplemented with the cytokine cocktail of: 20 ng/ml IL-3, 250 ng/ml IL-6, 10 ng/ml SCF, 250 ng/ml TPO and 100 ng/ml flt-3L and a 50% mixture of LPCM. LPCM is generated by obtaining a fresh human placenta from vaginal delivery and placing it in a sterile plastic container. The placenta is rinsed with an anticoagulant solution comprising phosphate buffered saline (Gibco-Invitrogen, Grand Island, N.Y.), containing a 1:1000 concentration of heparin (1% w/w) (American Pharmaceutical Partners, Schaumburg, Ill.). The placenta is then covered with a DMEM media (Gibco) in a sterile container such that the entirety of the placenta is submerged in said media, and incubated at 37° C. in a humidified 5% CO² incubator for 24 hours. At the end of the 24 hours, the live placenta conditioned medium (LPCM) is isolated from the container and sterile-filtered using a commercially available sterile 0.2 micron filter (VWR). Cells are expanded, checked for purity using CD34-specific flow cytometry and immunologically matched to recipients using a mixed lymphocyte reaction. Cells eliciting a low level of allostimulatory activity to recipient lymphocytes are selected for transplantation.

Cells are administered into 10 autistic patients intravenously at a concentration of 1.5 million CD34 cells per recipient on days 1, 3, and 5. Patients are also injected intravenously with endometrial regenerative cells extracted and propagated as described in Meng et al. Endometrial regenerative cells: a novel stem cell population. J Transl Med. 2007 Nov. 15; 5:57. Endometrial regenerative cells are administered at a concentration of 3 million cells intravenously on days 1, 3, and 5.

A statistically significant response in The Aberrant Behavior Checklist (ABC) score and the Vineland Adaptive Behavior Scale is observed in the patients receiving autologous cord blood but not placebo at the 1 month follow-up point

### Example 3

### Allogeneic Expanded Cord Blood CD34 Cell Therapy

20 children meeting the DSM-IV, ADI-R and ADOS-G criteria for autistic disorder are recruited into an experimental study. 10 patients serve as placebo controls whereas 10 receive active treatment, including the administration of allogeneic CD34 cord blood expand cells. The Aberrant Behavior Checklist (ABC) score and the Vineland Adaptive Behavior Scale are used in the selection of patients to enable the study to compare groups with similar characteristics.

10 patients are treated with 5 million CD34 cells prepared as described in Example 2. Administration is performed on days 1, 3, and 5. A statistically significant response in The Aberrant Behavior Checklist (ABC) score and the Vineland Adaptive Behavior Scale is observed in the patients receiving autologous cord blood but not placebo at the 1 month follow-up point.

One skilled in the art will appreciate that these methods, compositions, and cells are and may be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. It will be apparent to one skilled in the art that varying substitutions and modifications may be made to the invention disclosed herein without departing from the scope and spirit of the invention. Those skilled in the art recognize that the aspects and embodiments of the invention set forth herein may be practiced separate from each other or in conjunction with each other. Therefore, combinations of separate embodiments are within the scope of the invention as disclosed herein. All patents and publications mentioned in the specification are indicative of the levels of those skilled in the art to which the invention pertains. All patents and publications are herein incorporated by reference to the same extent as if each individual publication was specifically and individually indicated to be incorporated by reference.

While the teachings herein have predominately been directed to the treatment of autism, those with skill in the art can use the methods and kits provided herein to treat other suitable developmental disorders in patients, including but not limited to: Autism, Rett's Disorder, Childhood Disintegrative Disorder, and Asperger's Syndrome, and Pervasive Developmental Disorders Not Otherwise Specified (or PDDNOS).

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

What is claimed is:

1. A method of treating a pervasive developmental disorder comprising: providing a) a first cell capable of inhibiting inflammation and b) a second cell capable of stimulating angiogenesis; and administering said first and second cells either in sequence or concurrently into a patient in need thereof.

2. The method of claim 1, wherein said pervasive developmental disorder is selected from the group consisting of: Autism, Rett's Disorder, Childhood Disintegrative Disorder, and Asperger's Syndrome.

3. The method of claim 1, wherein said cell capable of inhibiting inflammation is a mesenchymal stem cell.

4. The method of claim 1, wherein said cell capable of inhibiting inflammation is activated in vivo by administering to said patient one or more agents capable of activating an anti-inflammatory response in said cell.

5. The method of claim 1, wherein said cell capable of stimulating angiogenesis acts by differentiating into cells of the patient's vasculature or by providing trophic support to cells of the patient's vasculature.

6. The method of claim 1, wherein said cell capable of stimulating angiogenesis is selected from the group consisting of: a) a cord blood derived CD34 cell, b) a cord blood mononuclear cell, c) a placental matrix mesenchymal cell, d) a mesenchymal cell, e) an endothelial progenitor cell, f) a monocytic cell, g) a bone marrow derived CD34 cell, h) a cord blood derived CD133 cell, and i) a bone marrow derived CD133 cell.

7. The method of claim 6, wherein an angiogenic agent is further administered to said patient to augment the angiogenic activity of said cell capable of stimulating angiogenesis.

8. A kit useful for the treatment of a pervasive developmental disorder in a patient in need thereof comprising: a) a cell capable of stimulating angiogenesis in said patient; and b) a cell capable of inhibiting inflammation in said patient.

9. The kit of claim 8, wherein said cell capable of stimulating angiogenesis is a cord blood derived CD34 cell.

10. The kit of claim 8, wherein said cell capable of inhibiting inflammation is a mesenchymal stem cell.

11. The kit of claim 10, wherein said mesenchymal stem cell is collected from a source selected from the group consisting of: a) bone marrow, b) placental matrix, c) adipose tissue, d) menstrual blood, e) endometrium, f) muscle, g) circulating blood, and h) cord blood.

12. The kit of claim 10, wherein said mesenchymal stem cell is allogeneic.

13. The kit of claim 8, wherein said cell capable of stimulating angiogenesis is allogeneic.

14. The kit of claim 8, further comprising instructions for administering said cell capable of stimulating angiogenesis and said cell capable of inhibiting inflammation to treat said pervasive developmental disorder in said patient.

15. A method of treating a pervasive developmental disorder comprising: concurrently stimulating an anti-inflammatory process and a pro-angiogenic process in a patient in need thereof.

16. The method of claim 15, wherein said stimulating an anti-inflammatory process is accomplished by administering a mesenchymal stem cell to said patient.

17. The method of claim 15, wherein said stimulating a pro-angiogenic process is accomplished by administering a cord blood derived CD34 cell to said patient.

18. The method of claim 15, wherein stimulation of an anti-inflammatory process is accomplished by administration of a non-cellular pharmaceutical anti-inflammatory agent.

19. The method of claim 15, wherein stimulation of a pro-angiogenic process is accomplished by administration of a pro-angiogenic, non-cellular pharmaceutical.

20. The method of claim 15, wherein a combination of an anti-inflammatory non-cellular pharmaceutical is provided with a pro-angiogenic cell.
